(19) United States
(12) Patent Application Publication
(10) Pub. No.: US 2025/0154442 A1
(43) Pub. Date: May 15, 2025

(54) SYSTEM FOR LIGHT STIMULATION OF A BIOLOGICAL SAMPLE

(71) Applicant: Commissariat à l'Energie Atomique et aux Energies Alternatives, Paris (FR)

(72) Inventor: Pierre BLEUET, Grenoble cedex 09 (FR)

(73) Assignee: Commissariat à l'Energie Atomique et aux Energies Alternatives, Paris (FR)

(21) Appl. No.: 18/940,933

(22) Filed: Nov. 8, 2024

(30) Foreign Application Priority Data
Nov. 9, 2023 (FR) 2312260

(51) Int. Cl. C12M 1/00; C12M 1/22; C12M 1/34

(52) U.S. Cl. CPC C12M 31/08; C12M 23/10; C12M 41/08; C12M 41/46

(57) ABSTRACT

A system for light stimulation of a biological sample includes a light source adapted to emit an input light beam, and a closed chamber to receive at least one container in which the biological sample is placed. The system includes an optical device, and an optical guide connected on one side to the light source so as to receive the input light beam and on the other side to the optical device, the optical guide being arranged to guide the input light beam emitted by the light source as far as the optical device and to emit an output light beam. The optical device is configured to generate, from the output light beam, a parallel stimulating light beam, with a predetermined constant section.

### TECHNICAL FIELD OF THE INVENTION

The present invention relates to a system for light stimulation of a biological sample.

### PRIOR ART

Photobiomodulation or phototherapy are techniques that are increasingly used for healing or slowing the progress of a pathology using light. Photobiomodulation is used for a wide variety of applications, in the treatment of neurodegenerative diseases, fibromyalgia or pain.

To assess the effectiveness of photobiomodulation on a biological sample such as cells, it is known practice to place Petri dishes containing the cells on a light panel, this panel being itself placed in a closed chamber so that it is in a temperature-controlled (for example at 37° C.) and humidity-controlled (80-90% humidity) environment. The light panel thus generates a stream of light through each Petri dish placed on the panel.

However, in practice, this configuration has a number of drawbacks:
During long tests, lasting several hours, the electronic board of the light panel has a tendency to heat up, which increases the temperature inside the chamber, possibly leading to deterioration of the cells; this means that the illumination panel has to be placed on a cooling panel (for example with circulation of temperature-controlled water) to dissipate the heat generated by the electronic board. It becomes difficult to control the temperature inside the chamber and manage the circulation of water in the cooling panel and the operating point becomes unstable. Since the set temperature of the cooling panel is low in order to discharge the heat efficiently and since the humidity in the chamber is set at around 80%, the dew point is exceeded and condensation then becomes an issue (oxidation, electrical hazards, humidity level).
The entire illumination panel emits light, which is not necessary since only the Petri dishes need to be illuminated; this results in reflections of light, which can influence the biological study, and overconsumption, which can have an effect on the temperature inside the chamber.
The light-emitting diodes of the illumination panel have been found to age more rapidly under the effect of temperature. For example, there may be a loss of light efficiency of 10% per annum under intensive use, thus necessitating frequent replacement of the illumination panel in order to maintain acceptable performance levels.

Stimulation solutions are described in patent applications CN112899157 and JP2006174764.

The aim of the invention is to propose a system used for the light stimulation of a biological sample, this system having a configuration making it possible to overcome the drawbacks of the prior art.

### SUMMARY OF THE INVENTION

This aim is achieved by a system for light stimulation of a biological sample, comprising:
A light source adapted to emit a light beam, referred to as the input light beam,
A closed chamber intended to receive at least one container in which the biological sample is placed,
The light source being placed outside the chamber,
The system including an optical device placed inside the chamber,
The system including an optical guide connected on one side to said light source so as to receive said input light beam and on the other side to said optical device, the optical guide being arranged to guide the input light beam emitted by the light source as far as the optical device and to emit a light beam, referred to as the output light beam, intended for the optical device,
Said optical device being configured to generate, from said output light beam, a parallel stimulating light beam for stimulating the biological sample, with a predetermined constant section.

According to a particular feature, the optical device includes an optical assembly configured to generate a divergent light beam from the output light beam and an off-axis parabolic mirror positioned relative to said optical assembly so as to reflect said divergent light beam and form said parallel stimulating light beam.

According to another particular feature, the system includes means for adjusting the position of said optical assembly relative to the position of the off-axis parabolic mirror.

According to another particular feature, the system includes means for adjusting the position of the off-axis parabolic mirror relative to the position of said optical assembly.

According to another particular feature, the system includes means for adjusting the orientation of said optical assembly relative to the off-axis parabolic mirror.

According to another particular feature, the system includes means for adjusting the orientation of the off-axis parabolic mirror relative to said optical assembly.

According to another particular feature, the light source is a regulated laser source.

According to another particular feature, the system includes a mask arranged at the outlet of the optical device for shaping the section of the parallel stimulating light beam.

According to another particular feature, the optical guide includes a splitter, arranged to split said input light beam into said output light beam and a second output light beam, the system including a second optical device for shaping the second output light beam, configured to generate a second stimulating light beam.

According to another particular feature, the closed chamber is an incubator.

The invention also relates to a method for light stimulation of a biological sample, implemented using the system as defined above, the method including the following steps:
Positioning, in said closed chamber, a container in which is placed said biological sample to be stimulated, said container including an illumination window having an illumination section through which a light beam can penetrate, this light beam being a parallel stimulating light beam for stimulating the biological sample,
Configuring the optical device so as to obtain the parallel stimulating light beam with a constant section having a surface area greater than or equal to the illumination section of the illumination window of the container,
Activating said light source to emit the input light beam inside the optical guide and obtain the output light beam to be applied to the optical device for shaping this beam.

According to a particular feature, the container is a Petri dish.

### BRIEF DESCRIPTION OF THE FIGURES

Further features and advantages will become clear from the detailed description below, which is provided with reference to the attached drawings, in which:

FIG. 1 depicts, schematically, the system for light stimulation of a biological sample, in accordance with the invention;

FIG. 2 schematically shows the architecture of the last part of the stimulation system of the invention;

FIG. 3 shows the operating principle of the stimulating light beam that illuminates the container containing the biological sample;

FIG. 4 shows, in a perspective view, an example of an embodiment of the optical device used in the system of the invention;

FIG. 5 shows, schematically, an advantageous embodiment of the system for light stimulation of a biological sample in accordance with the invention;

FIG. 6 shows, schematically, an alternative embodiment of the optical device used in the system of the invention.

### DETAILED DESCRIPTION OF AT LEAST ONE EMBODIMENT

The invention relates to the light stimulation of a biological sample ECH. “Biological sample ECH” means, for example, cells of a living being, but also any other plant or animal substance for which light stimulation could have an effect.

The invention relates more particularly to cell cultures, more specifically to the influence of light on cell cultures and to the monitoring of the effect of light on cell cultures. The terms “light” or “light beam” used below mean any electromagnetic radiation with a wavelength ranging from the ultraviolet to the far infrared, including visible light.

### Container

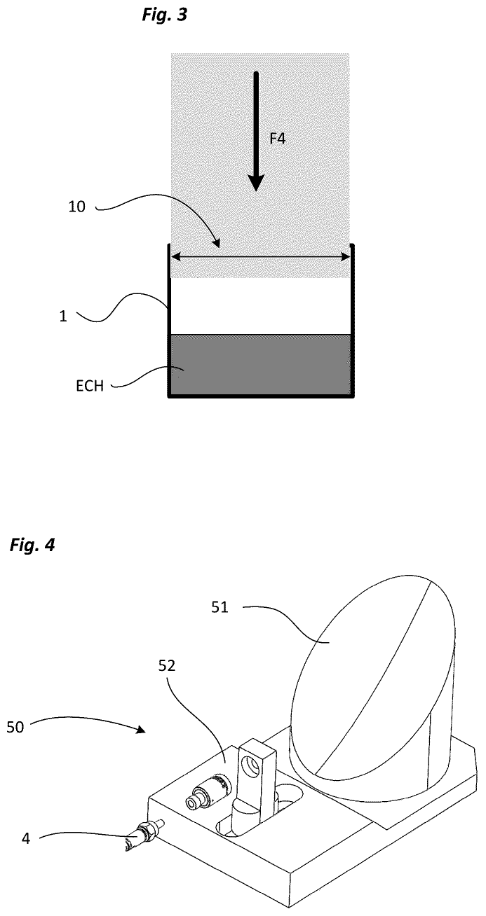

### FIG. 3

The biological sample ECH is placed in a container 1. This container 1 includes a side wall and a bottom wall and may be open at the top or closed by a wall that is transparent to one or more wavelengths of the light beam emitted by the light source of the system. The area through which the light beam passes defines an illumination window 10 for illuminating the biological sample ECH. This illumination window 10 has a cross section delimiting said area through which the light beam passes. This cross section is advantageously circular. However, as will be seen below, this section could be different and take any other form, by virtue of a slight adaptation of the system.

The container 1 is for example produced in the form of a Petri dish.

The system for light stimulation of the invention mainly includes:
A light source 2;
A chamber 3;
An optical guide 4;
An optical device 5.

### Light Source

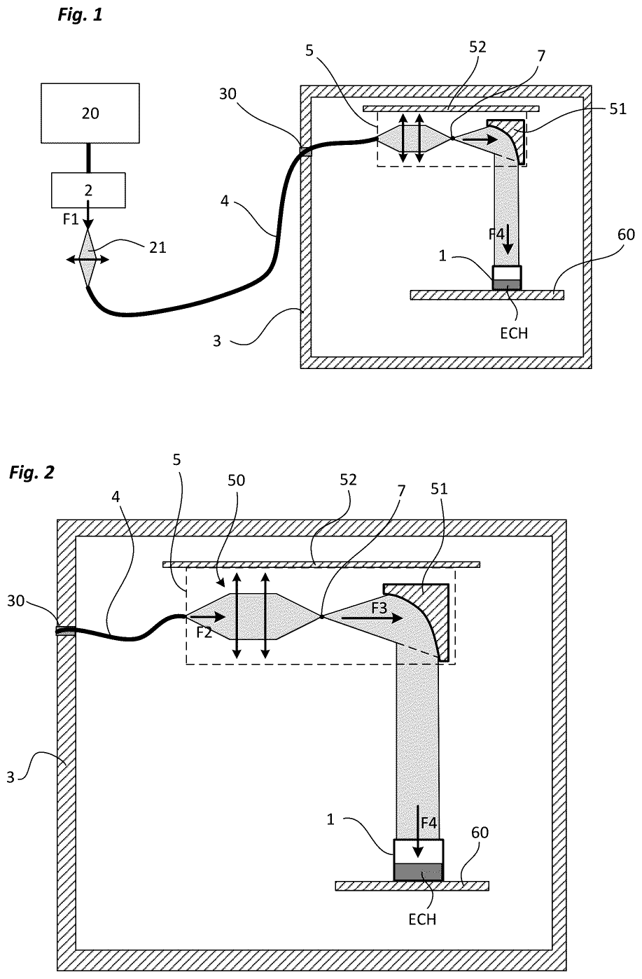

### FIG. 1

### FIG. 2

The system includes at least one light source 2. This light source 2 is supplied with power so as to generate a light beam, referred to here as the input light beam F1.

The light source 2 may consist of one or more light-emitting diodes or advantageously of at least one laser diode.

The light source 2 is advantageously of regulated intensity.

In the field of photobiomodulation, the light source 2 is selected to emit at a wavelength of between 600 nm and 1000 nm.

In an example of an embodiment, use is made of a control unit 20 which makes it possible to supply current to a laser diode with a wavelength of 670 nm, emitting a maximum of 1.3 W. The current delivered by the power supply unit is of the order of 1 ampere. This same control unit 20 makes it possible to regulate the temperature of the laser diode, which is mounted on a specific mechanism.

Note that the laser diode emits the input light beam F1 which is highly divergent and that it is thus necessary to refocus the beam with a set of lenses 21 in order to guide the light efficiently towards an optical guide (see below), the numerical aperture of the set of lenses being less than the numerical aperture of the optical guide 4 for optimal coupling.

According to the invention, the light source 2 is positioned outside the closed chamber 3.

### Closed Chamber

### FIG. 1

### FIG. 2

The system includes a closed chamber 3 in which said container 1 receiving the biological sample ECH is placed.

This chamber 3 is sealed closed. In the case of a cell culture, the internal space of the chamber 3 is advantageously temperature- and humidity-controlled so as to obtain the optimal conditions for cell growth. By way of example, the temperature may be set at 37° C. and the humidity at around 80%. A specific control unit may be configured to manage the temperature and the humidity inside the chamber 3.

The chamber 3 is for example an incubator. This incubator may include at least one sealed passage 30 formed through one of its walls, this passage being adapted for the insertion therein of an optical guide 4.

Advantageously, the chamber is closed in such a way as to have walls that are completely opaque to external light and to form an internal volume which is as dark as possible.

Advantageously, the internal surface of the chamber may be covered with a material that absorbs the photobiomodulation wavelengths, so as to prevent secondary reflections that would disrupt stimulation.

### Optical Guide

### FIG. 1

### FIG. 2

To route the input light beam F1 emitted by the light source 2 towards the inside of the chamber 3, the system includes an optical guide 4, for example an optical fibre.

As stated above, in the case of a light source of laser diode or light-emitting diode type that emits a highly divergent beam, it may be necessary to use a set of lenses at the inlet of the optical guide 4 and to ensure that the numerical aperture of the lens is less than the numerical aperture of the optical guide for optimal coupling.

The optical guide 4 penetrates through the chamber 3, via the dedicated passage 30. The input light beam F1 emitted by the light source 2, located outside the chamber, is thus routed inside the chamber via the optical guide 4.

Note that the optical guide 4 has flexibility characteristics allowing it to be slightly curved and manipulated, facilitating the overall installation of the system.

### Optical Device

### FIG. 1

### FIG. 2

### FIG. 3

### FIG. 4

The optical device 5 is positioned inside the chamber 3, at the outlet of the optical guide 4. Its function is to recover the light beam obtained at the outlet of the optical guide and referred to as the output light beam F2, in order to shape this beam.

The optical device 5 is configured to shape the output light beam F2 so as to obtain the desired parallel stimulating light beam F4 for stimulating the biological sample ECH.

According to the invention, the aim is to obtain a parallel stimulating light beam F4 having a constant section, advantageously circular, over its entire length, between the optical device 5 and the container 1 receiving the biological sample ECH.

The circular section of the beam F4 obtained is advantageously greater than or equal to the circular section of the illumination window 10 of the container 1.

The optical device 5 includes an optical assembly 50 configured to generate a divergent light beam F3 from the output light beam F2 and an off-axis parabolic mirror 51 intended to receive said divergent light beam F3 so as to reflect the latter and shape said parallel stimulating light beam F4.

The optical assembly 50 may be composed of two convergent lenses positioned in series in a suitable manner and not adjoining. At the outlet of the second lens, the beam obtained converges first towards a source point that thus forms a secondary light source 7. The optical assembly 50 is designed in such a way that this secondary source 7 is positioned at the focal length of the off-axis parabolic mirror 51, which makes it possible to ensure that the beam F4 exiting the mirror 51 is parallel and not divergent. This secondary source 7 is divergent towards the off-axis parabolic mirror 51. The optical assembly 50 thus makes it possible to create this intermediate divergent secondary source 7 from the output light beam F2.

As a reminder, an off-axis parabolic mirror 51 is a known optical device, with a curved reflective surface, having a parabolic profile. It has the capacity to reflect a parallel stream of light from a divergent light source.

At the outlet of the optical guide 4, the optical assembly 50 and its position must be selected such that the divergence of the light beam F3 generated is sufficient to illuminate a large part of the off-axis parabolic mirror 51 and to ensure that a reflected light beam F4 used for the stimulation is obtained which has a constant section that is sufficient relative to the section of the illumination window 10 of the container 1. In other words, the optical assembly 50 must be capable of focusing the light beam and forming the corresponding secondary source 7 at the focal length of the off-axis parabolic mirror.

By way of example, an off-axis parabolic mirror measuring 3 inches (76.2 mm) will make it possible to illuminate a conventional Petri dish.

The parallel stimulating light beam F4 generated makes it possible to have homogeneous illumination over the entire illumination window 10 of the container 1, and to be able to light up only said container 1, on the condition, of course, that the diameter of the off-axis parabolic mirror 51 is selected to be greater than or equal to the diameter of the illumination window 10 (for example the diameter of the Petri dish).

Advantageously, as shown in FIG. 4, the optical device 5 may be made in one optical piece including a support 52 on which the optical assembly 50 and the off-axis parabolic mirror 51 are arranged. The optical assembly 50 may notably be mounted slidingly in said support 52 to allow adjustment of its position relative to the off-axis parabolic mirror 51. Likewise, it would also be possible to adjust the position of the off-axis parabolic mirror.

Moreover, means for adjusting the orientation of the optical assembly 50 and/or of the off-axis parabolic mirror 51 could be provided so as to be able to orient one with respect to the other.

If the section of the illumination window 10 of the container is not circular or if the parallel stimulating light beam F4 has a section which is too big relative to that of the illumination window 10 of the container, the system may include a mask for modifying the section of the parallel stimulating light beam F4, so that it matches as closely as possible the shape of the illumination window 10 of the container. In this case, it will be appreciated that the invention does not necessarily apply to the case in which the illumination window 10 of the container has a circular section.

### Arrangement of the System

### FIG. 1

### FIG. 2

### FIG. 3

### FIG. 4

As stated above, the light source 2 is positioned outside the chamber 3.

The optical guide 4 is connected to the light source so as to route the input light beam emitted by the source towards the inside of the chamber.

On the other side, the optical guide 4 is connected to the optical assembly 50 of the optical device 5. The light beam F2 obtained at the outlet of the optical guide 4 is sent onto the optical assembly 50.

Optical connectors are provided for connecting each end of the optical guide 4.

The optical assembly 50 makes it possible to generate the divergent source creating the divergent light beam F3 in the direction of the off-axis parabolic mirror 51.

The off-axis parabolic mirror 51 receives the divergent light beam F3 and reflects the latter so as to obtain the desired parallel stimulating light beam F4, intended for the container 1.

The container 1 is for example placed on a first flat support 60 present in the closed chamber 3, its illumination window 10 then being parallel to this first support.

The optical device may be attached by means of its support 52 to a plate mounted parallel to the support 60 supporting the container 1, such that the off-axis parabolic mirror 51 emits the parallel stimulating light beam F4 in a direction perpendicular to the illumination window 10. This plate could support several optical devices 5 in parallel, so as to be able to treat several samples in parallel.

As the light beam is routed towards the optical device 5 by the optical guide 4, it is not necessary to specifically orient the light source 2 relative to the optical device 5, which facilitates the installation of the system.

### ALTERNATIVE EMBODIMENTS

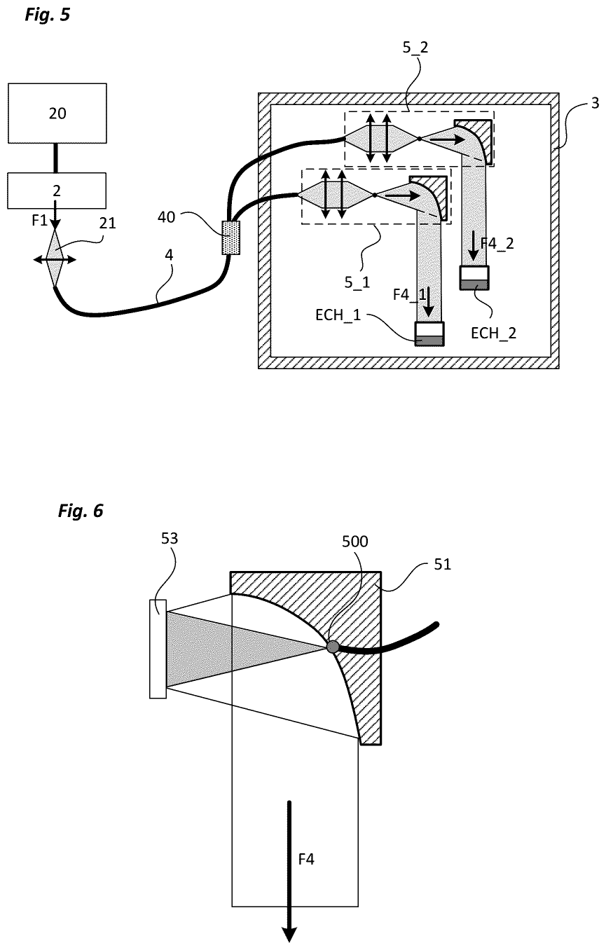

### FIG. 5

### FIG. 6

An advantageous alternative embodiment illustrated by FIG. 5 makes it possible, using a single light source 2, to light up several containers 1. To this end, the system incorporates a splitter 40 arranged on the optical guide 4 and having the function of splitting the input light beam F1 emitted by the light source 2 at the inlet of the optical guide 4 into several output light beams. The system then includes several optical devices 5_1, 5_2 in parallel, each having the task of generating a different parallel stimulating light beam F4_1, F4_2 intended for a specific container 1. Thus, several biological samples ECH1, ECH2 are optically stimulated in parallel.

Another alternative embodiment shown in FIG. 6 pertains to the production of the optical device. According to this alternative embodiment, the divergent source 500 (created by the optical assembly 50) is positioned in the off-axis parabolic mirror 51 (which is thus pierced) and a reflection mirror 53 (flat mirror) is added which thus reflects the incident divergent beam towards the off-axis parabolic mirror 51. An optical fibre may be used to convey the light as far as the point forming the divergent source 500. This solution is more difficult to install but also more compact.

### ADVANTAGES

The invention thus has many advantages, including:
It makes it possible to light up an area of interest in an identical manner at all points so that each biological cell is stimulated with the same intensity and the same incidence of the light wave;
It makes it possible to solve the thermal problems encountered in the closed chamber, by moving the light source and the control thereof to the outside;
It makes it possible to create a homogeneous beam which perfectly matches the area to be lit up;
It makes it possible to light up several biological samples in parallel, with identical light beams;
It is simple to implement and can offer options for adjustment taking into account the dimensions of the areas to be lit up.

1. A system for light stimulation of a biological sample, comprising:
a light source configured to emit an input light beam, and
a closed chamber to receive a container in which the biological sample is placed, wherein
the light source is placed outside the chamber,
the system includes an optical device placed inside the chamber,
the system includes an optical guide connected on one side to said light source so as to receive said input light beam and on the other side to said optical device, the optical guide being arranged to guide the input light beam emitted by the light source as far as the optical device and to emit an output light beam to the optical device, and
optical device is configured to generate, from said output light beam, a parallel stimulating light beam for stimulating the biological sample, with a predetermined constant section.

2. The system according to claim 1, wherein the optical device includes an optical assembly configured to generate a divergent light beam from the output light beam, and an off-axis parabolic mirror positioned relative to said optical assembly so as to reflect said divergent light beam and form said parallel stimulating light beam.

3. The system according to claim 2, further comprising means for adjusting a position of said optical assembly relative to a position of the off-axis parabolic mirror.

4. The system according to claim 2, further comprising means for adjusting a position of the off-axis parabolic mirror relative to a position of said optical assembly.

5. The system according to claim 2, further comprising means for adjusting an orientation of said optical assembly relative to the off-axis parabolic mirror.

6. The system according to claim 2, further comprising means for adjusting an orientation of the off-axis parabolic mirror relative to said optical assembly.

7. The system according to claim 1, wherein the light source is a regulated laser source.

8. The system according to claim 1, further comprising a mask arranged at an outlet of the optical device for shaping the section of the parallel stimulating light beam.

9. The system according to claim 1, wherein the optical guide includes a splitter, arranged to split said input light beam into said output light beam and a second output light beam, and the system includes a second optical device for shaping the second output light beam, configured to generate a second stimulating light beam.

10. The system according to claim 1, wherein the closed chamber is an incubator.

11. A method for light stimulation of the biological sample, implemented using the system as defined in claim 1, the method comprising:
positioning, in said closed chamber, the container in which is placed said biological sample to be stimulated, said container including an illumination window having an illumination section through which a light beam can penetrate, the light beam being a parallel stimulating light beam for stimulating the biological sample,
configuring the optical device so as to obtain the parallel stimulating light beam with a constant section having a surface area greater than or equal to the illumination section of the illumination window of the container, and
activating said light source to emit the input light beam inside the optical guide and obtain the output light beam to be applied to the optical device for shaping the beam.

12. The method according to claim 11, wherein the container is a Petri dish.
